(19) United States
(12) Patent Application Publication
(10) Pub. No.: US 2025/0072844 A1
(43) Pub. Date: Mar. 6, 2025

(54) X-RAY CT APPARATUS

(71) Applicant: FUJIFILM Corporation, Tokyo (JP)

(72) Inventor: Masanori KAKIMOTO, Chiba (JP)

(73) Assignee: FUJIFILM Corporation, Tokyo (JP)

(21) Appl. No.: 18/817,197

(22) Filed: Aug. 27, 2024

(30) Foreign Application Priority Data
Aug. 29, 2023 (JP) 2023-139033

(51) Int. Cl. A61B 6/03

(52) U.S. Cl. CPC A61B 6/035

(57) ABSTRACT

Provided is an X-ray CT apparatus capable of suppressing an entry of foreign matter into an inside of a bearing without incurring a cost. The X-ray CT apparatus of an embodiment of the present invention includes a fixed frame, a rotation frame, and a bearing, in which the bearing includes an outer ring connected to the fixed frame and an inner ring connected to the rotation frame, and the fixed frame includes a seal portion that seals the bearing.

### CROSS-REFERENCE TO RELATED APPLICATION

The present application claims priority under 35 U.S.C. § 119(a) to Japanese Patent Application No. 2023-139033 filed on Aug. 29, 2023, which is hereby expressly incorporated by reference, in its entirety, into the present application.

### BACKGROUND OF THE INVENTION

### 1. Field of the Invention

The present invention relates to an X-ray CT apparatus, and more particularly, to an X-ray CT apparatus comprising a gantry including a turn plate.

### 2. Description of the Related Art

JP2010-5319A discloses an X-ray computed tomography (CT) apparatus comprising a slip ring, in which the X-ray computed tomography apparatus prevents abrasion powder caused by contact between the slip ring and a brush from entering an inside of the X-ray computed tomography apparatus.

According to JP2010-5319A, a plurality of fins are provided on a rotation side of the X-ray computed tomography apparatus, and these fins are rotated with the rotation of the rotation side to generate an air flow. The air flow releases the abrasion powder to an outside of the X-ray CT apparatus and prevents the abrasion powder from scattering toward an X-ray tube, a support bearing, and the like.

### SUMMARY OF THE INVENTION

However, the X-ray CT apparatus of JP2010-5319A has a configuration in which the plurality of fins are provided on the rotation side (a rotation frame side), which poses problems of a complicated structure of a rotation frame and a cost increase.

In addition, it is also conceivable to employ, as the support bearing of the rotation frame, a bearing provided with a seal structure (for example, a labyrinth seal structure) capable of preventing the entry of foreign matter such as abrasion powder. However, the bearing provided with such a seal structure is not inexpensive. Therefore, in this case as well, there is a problem of a cost increase.

The present invention has been made in order to solve the above-described problems, and an object of the present invention is to provide an X-ray CT apparatus capable of suppressing an entry of foreign matter into an inside of a bearing without incurring a cost.

According to a first aspect, there is provided an X-ray CT apparatus comprising: a fixed frame; a rotation frame on which mounted objects including an X-ray source and an X-ray detector are mounted; and a bearing that supports the rotation frame to be rotatable with respect to the fixed frame, in which the bearing includes an outer ring connected to the fixed frame and an inner ring connected to the rotation frame, and the fixed frame includes a seal portion that seals the bearing.

According to a second aspect, in the X-ray CT apparatus, it is preferable that the seal portion is configured integrally with the fixed frame.

According to a third aspect, in the X-ray CT apparatus, it is preferable that the seal portion is an end part of the fixed frame connected to the outer ring, the end part projecting to face at least one of two open parts on both sides of the bearing in a rotation axis direction.

According to a fourth aspect, in the X-ray CT apparatus, it is preferable that a driving unit including a pulley that rotationally drives the rotation frame is further provided, a recessed portion is provided between the inner ring and the pulley, and the seal portion is inserted into the recessed portion with a gap therebetween, and the seal portion and the recessed portion constitute a labyrinth seal.

According to a fifth aspect, in the X-ray CT apparatus, it is preferable that a recessed portion is provided between the inner ring and the rotation frame, and the seal portion is inserted into the recessed portion with a gap therebetween, and the seal portion and the recessed portion constitute a labyrinth seal.

According to the aspects of the present invention, it is possible to suppress the entry of foreign matter into the inside of the bearing without incurring a cost.

### BRIEF DESCRIPTION OF THE DRAWINGS

FIG. 1 is an external perspective view of an X-ray CT apparatus of an embodiment.

FIG. 2 is a front view of a turn plate with a cover removed from the gantry of FIG. 1.

FIG. 3 is a front view of a state in which a rotation frame is removed from the turn plate of FIG. 2.

FIG. 4 is a cross-sectional view taken along line 4-4 of the turn plate of FIG. 2.

FIG. 5 is an enlarged cross-sectional view of a main part showing a seal structure in a rotation support part of the rotation frame.

### DESCRIPTION OF THE PREFERRED EMBODIMENTS

Hereinafter, an X-ray CT apparatus of an embodiment of the present invention will be described with reference to the accompanying drawings.

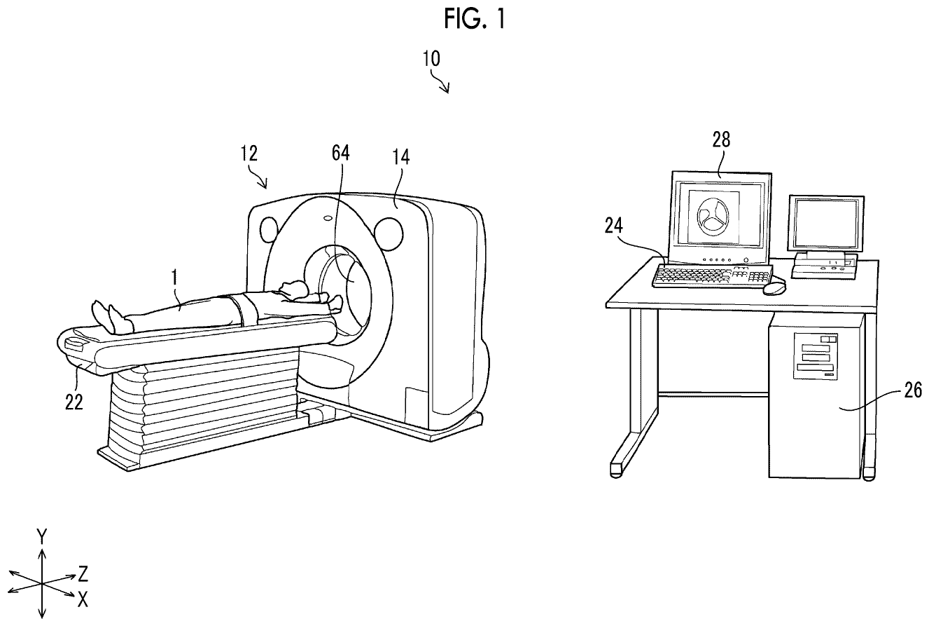

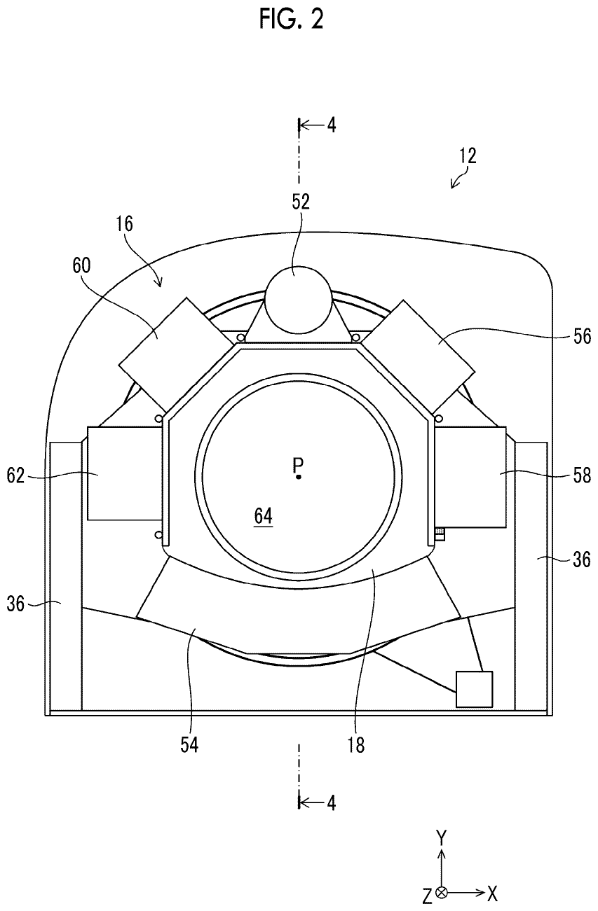

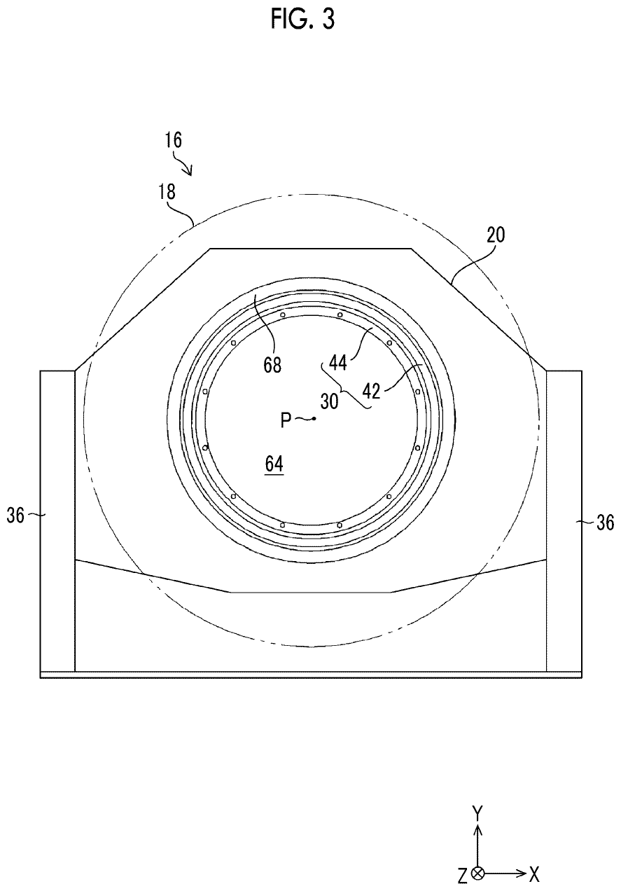

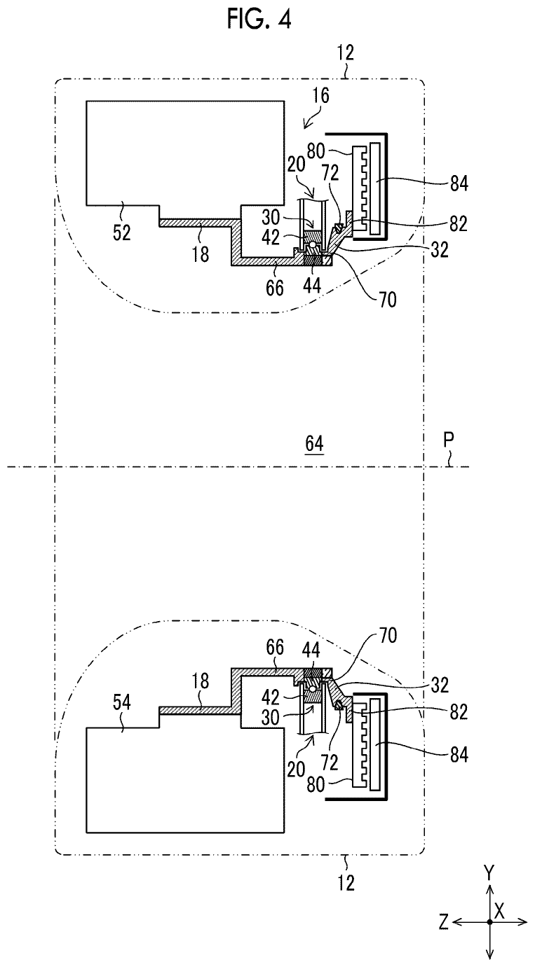

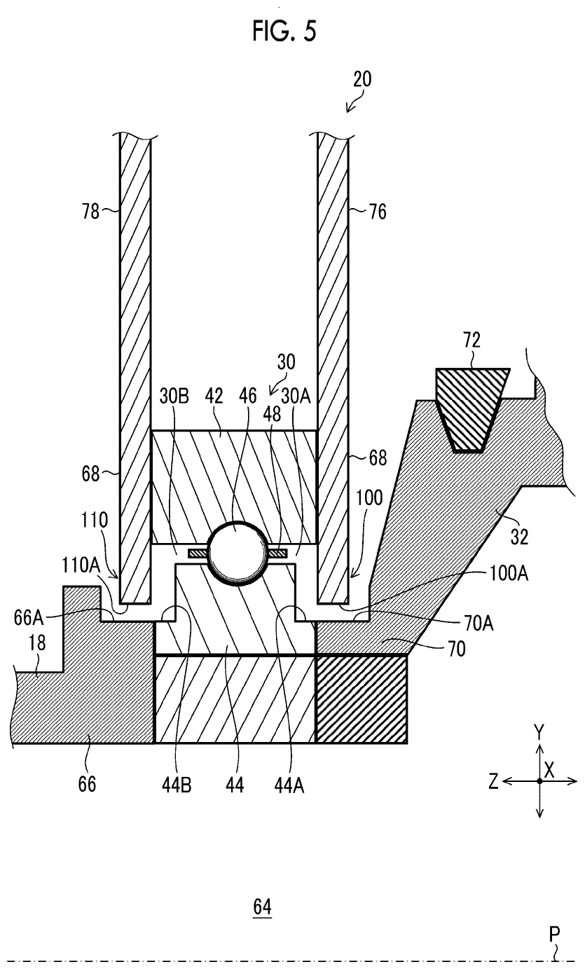

FIGS. 1 to 4 show a structure of the X-ray CT apparatus of the embodiment. FIG. 1 is an external perspective view of an X-ray CT apparatus 10, FIG. 2 is a front view of a turn plate 16 with a front cover 14 removed from a gantry 12 of FIG. 1, and FIG. 3 is a front view of a structure including a fixed frame 20 with a rotation frame 18 removed from the turn plate 16 of FIG. 2. FIG. 4 is a cross-sectional view taken along line 4-4 of the turn plate 16 of FIG. 2. FIG. 5 is an enlarged cross-sectional view of a main part showing a seal structure in the rotation support part of the rotation frame 18.

The three-dimensional coordinate system shown in FIGS. 1 to 5 shows an example of the definition of directions in the X-ray CT apparatus 10. An X axis, a Y axis, and a Z axis of the three-dimensional coordinate system are examples, and the present invention is not limited thereto. For ease of understanding in the following description, the X axis, the Y axis, and the Z axis in the X-ray CT apparatus 10 are defined in the same directions in all the drawings. A Z axis direction is a body axis direction of a subject 1 in a case where the subject 1 is placed on a patient table 22. AY axis direction is an up-down direction of the subject 1 and is a direction parallel to a direction of gravity. An X axis direction is a left-right direction of the subject 1 and is a horizontal direction orthogonal to the direction of gravity.

As shown in FIG. 1, the X-ray CT apparatus 10 comprises the gantry 12, the patient table 22 on which the subject 1 is placed, an input device 24, an image calculation device 26, and a display device 28.

The gantry 12 includes the front cover 14 and the turn plate 16 disposed inside the front cover 14. The turn plate 16 comprises the rotation frame 18, the fixed frame 20 that supports the rotation frame 18 from a rear side via a bearing 30, and a pulley 32 for rotationally driving the rotation frame 18. In addition, the turn plate 16 has a tilt mechanism that rotates the rotation axis direction in an inclined state with respect to a principal plane of a tabletop of the patient table 22. The turn plate 16 is supported by a stand 36.

As shown in FIGS. 4 and 5, in the present example, a ball bearing is applied as the bearing 30 that supports the rotation frame 18 to be rotatable with respect to the fixed frame 20.

The bearing 30 is composed of an outer ring 42, an inner ring 44, a ball 46 that is a rolling body, and a holder 48 (also referred to as a retainer) that holds the ball 46.

In addition, the bearing 30 includes two open parts 30A and 30B on both sides of the bearing 30 in a direction of a rotation axis P (which is the same as the rotation axis of the rotation frame 18). That is, the bearing 30 of the present example is a bearing having a basic structure in which a seal structure is not provided in the bearing 30 itself. It should be noted that, in the present example, the ball bearing is applied as the bearing 30, but the present invention is not limited thereto, and for example, a roller bearing may be applied. The bearing 30 is an example of a bearing of the embodiment of the present invention.

As shown in FIG. 2, on the rotation frame 18, mounted objects such as an X-ray tube 52, an X-ray detection unit 54, a cooling device 56, a controller 58, and a high-voltage generation units 60 and 62 are mounted along a circumferential direction centered on the rotation axis P.

The rotation frame 18 rotates around the subject 1 by power of a driving unit, which will be described below. In the rotation frame 18, the X-ray detection unit 54 is disposed to face the X-ray tube 52. The X-ray detection unit 54 detects X-rays transmitted through the subject 1 to measure a spatial distribution of the transmitted X-rays. The rotation frame 18 is an example of a rotation frame of the embodiment of the present invention. The X-ray tube 52 is an example of an X-ray source of the embodiment of the present invention. The X-ray detection unit 54 is an example of an X-ray detector of the embodiment of the present invention.

The image calculation device 26 shown in FIG. 1 performs CT image reconstruction by performing calculation processing on measurement data of the X-ray detection unit 54. The display device 28 displays a CT image created by the image calculation device 26. The input device 24 accepts an input of an imaging condition or the like from an operator.

As shown in FIGS. 3 and 4, the fixed frame 20 is disposed on a rear side of the rotation frame 18. The fixed frame 20 is configured in, for example, a polygonal shape in a front view in a case of being viewed from the Z axis direction.

The fixed frame 20 is provided with a circular opening part 64 at the center. A cylindrical sleeve 66 of the rotation frame 18 is disposed in the opening part 64, and the sleeve 66 is attached to the fixed frame 20 via the bearing 30.

Specifically, as shown in FIG. 5, the opening part 64 is defined by a circular opening edge portion 68 which is an end part of the fixed frame 20. The outer ring 42, which is a fixed side of the bearing 30, is connected to the opening edge portion 68, and the sleeve 66 of the rotation frame 18 is connected to the inner ring 44, which is the rotation side of the bearing 30. As a result, the rotation frame 18 is supported to be rotatable with respect to the fixed frame 20 via the bearing 30.

The fixed frame 20 of the present example has a double plate-like body structure in the Z axis direction. Inner peripheral edge portions of two plate-like bodies 76 and 78 constituting the fixed frame 20 are formed as the opening edge portion 68, and the outer ring 42 is fixed to the two plate-like bodies 76 and 78. The fixed frame 20 is an example of a fixed frame of the embodiment of the present invention.

As shown in FIGS. 4 and 5, the conical pulley 32 is disposed from an inner side of the opening part 64 of the fixed frame 20 toward the rear side of the fixed frame 20.

A small-diameter side end part 70 of the pulley 32 is connected to the inner ring 44 of the bearing 30 by a plurality of bolts (not shown). Consequently, the pulley 32 and the rotation frame 18 are connected to each other via the inner ring 44 of the bearing 30. As a result, in a case where the pulley 32 is rotationally driven, a rotational force thereof is transmitted to the rotation frame 18 via the inner ring 44, and the rotation frame 18 is rotated about the rotation axis P.

A motor (not shown) is disposed in the X-ray CT apparatus 10. A pulley is attached to a rotation axis of the motor, and a drive belt 72 is wound between the pulley and the pulley 32. The pulley 32 is rotated by the power of the motor, whereby the rotation frame 18 is rotationally driven about the rotation axis P.

The pulley 32, the above-described motor, the drive belt 72, and the like constitute a driving unit of the embodiment of the present invention. The pulley 32 is an example of a pulley provided in the above-described driving unit.

Meanwhile, as shown in FIG. 5, the opening edge portion 68 of the fixed frame 20 includes two seal portions 100 and 110 that seal the bearing 30. The seal portions 100 and 110 are configured integrally with the fixed frame 20 (two plate-like bodies 76 and 78).

In addition, the seal portions 100 and 110 are configured in a plate-like shape extending along the Y axis direction. In addition, the seal portions 100 and 110 are end parts (opening edge portion 68) of the fixed frame 20 connected to the outer ring 42 and are end parts projecting in the Y axis direction so as to face the open parts 30A and 30B of the bearing 30 in the Z axis direction.

In addition, among the seal portions 100 and 110, in the seal portion 100 on a side close to a slip ring 80 (refer to FIG. 4), which will be described below, an inner peripheral edge 100A of the seal portion 100 is inserted into a space between an L-like cutout portion 44A provided at a side part of the inner ring 44 on a pulley 32 side and an L-like cutout portion 70A provided at the end part 70 of the pulley 32, with a gap therebetween. These cutout portions 44A and 70A constitute a recessed portion of the embodiment of the present invention.

That is, the seal portion 100 is inserted into the recessed portion (cutout portions 44A and 70A) provided between the inner ring 44 and the pulley 32, with the gap therebetween. As a result, the seal portion 100 and the above-described recessed portion constitute a labyrinth seal.

In a case where the inner peripheral edge 100A is inserted into the above-described recessed portion, the gap (the gap in the Z axis direction and the Y axis direction) between the inner peripheral edge 100A and the recessed portion is set to about 1 mm, thereby forming a favorable labyrinth seal structure.

Meanwhile, in the seal portion 110, an inner peripheral edge 110A of the seal portion 110 is inserted into a space between a cutout portion 44B provided at a side part of the inner ring 44 on a rotation frame 18 side and a cutout portion 66A provided at the sleeve 66 of the rotation frame 18, with a gap therebetween. These cutout portions 44B and 66A constitute a recessed portion of the embodiment of the present invention.

That is, the seal portion 110 is inserted into the recessed portion (cutout portions 44B and 66A) provided between the inner ring 44 and the rotation frame 18, with a gap therebetween. As a result, the seal portion 110 and the above-described recessed portion constitute a labyrinth seal.

In a case where the inner peripheral edge 110A is inserted into the above-described recessed portion, the gap (gap in the Z axis direction and the Y axis direction) between the inner peripheral edge 110A and the recessed portion is set to about 1 mm, thereby forming a favorable labyrinth seal structure.

Hereinafter, the slip ring 80 will be described.

As shown in FIG. 4, the slip ring 80 having a plurality of conductor rings is fixed to a large-diameter side end part 82 of the pulley 32. A plurality of cables (not shown) are each connected to the slip ring 80. The plurality of cables pass through the inner side of the pulley 32 and reach the rotation frame 18 located on a front side of the bearing 30.

The plurality of conductor rings constituting the slip ring 80 have different diameters from each other and are arranged concentrically with respect to the rotation axis P. The plurality of conductor rings include a power supply conductor and a signal transmission conductor (not shown). The power supply conductor is disposed on an outer side (opposite to the rotation axis P) of the signal transmission conductor, as an example. The power supply conductor is configured to be wider than the signal transmission conductor.

A brush device 84 is disposed at a position facing the slip ring 80 in the Z axis direction. A power supply brush of the brush device 84 is brought into contact with the power supply conductor of the slip ring 80, and a signal transmission brush of the brush device 84 is brought into contact with the signal transmission conductor of the slip ring 80. The brush device 84 is fixed to a bracket (not shown) that is extended from the fixed frame 20.

In such an X-ray CT apparatus 10 including the slip ring 80 and the brush device 84, in order to prevent the rotation malfunction of the rotation frame 18, it is necessary to prevent the abrasion powder caused by the contact between the conductor ring and the brush from entering the inside of the bearing 30.

As a measure for preventing the entry of abrasion powder, it is conceivable to provide a plurality of fins on the rotation side (refer to JP2010-5319A) or to employ a bearing provided with a seal structure, but each of these measures poses a problem of increasing the cost of the apparatus.

In that respect, in the X-ray CT apparatus 10 of the embodiment, a configuration that solves the above-described problem is employed. Hereinafter, the above-described configuration will be described.

As shown in FIG. 5, the X-ray CT apparatus 10 of the embodiment comprises the fixed frame 20, the rotation frame 18, and the bearing 30, the bearing 30 includes the outer ring 42 connected to the fixed frame 20 and the inner ring 44 connected to the rotation frame 18, and the fixed frame 20 includes seal portions 100 and 110 that seal the bearing 30.

That is, the X-ray CT apparatus 10 of the embodiment employs a configuration in which the seal portions 100 and 110 are provided at the fixed frame 20. As a result, the X-ray CT apparatus 10 of the embodiment can suppress the entry of foreign matter into the inside of the bearing 30 without incurring a cost, as compared with the X-ray CT apparatus that comprises the plurality of fins on the rotation side or that employs the bearing provided with the seal structure.

In addition, in the X-ray CT apparatus 10 of the embodiment, the seal portions 100 and 110 are configured integrally with the fixed frame 20. Consequently, the X-ray CT apparatus 10 of the embodiment can save on component management costs, retrofitting costs, and the like, as compared with the X-ray CT apparatus having a configuration in which the seal portion is a separate body and retrofitted to the fixed frame. As a result, it is possible to reduce the manufacturing cost of the X-ray CT apparatus 10.

In addition, the X-ray CT apparatus 10 of the embodiment can suppress the entry of abrasion powder into the inside of the bearing 30 from the open parts 30A and 30B because the seal portions 100 and 110 are disposed to face both the two open parts 30A and 30B.

It should be noted that, in the embodiment, a configuration has been exemplified in which both the two open parts 30A and 30B are sealed by the seal portions 100 and 110, but the present invention is not limited thereto. For example, a configuration may also be employed in which one open part of the two open parts 30A and 30B is sealed by the seal portion. In this case, from the viewpoint of preventing the entry of abrasion powder, a configuration in which the open part 30A on the side close to the slip ring 80 among the open parts 30A and 30B is sealed by the seal portion 100 is preferable.

In addition, the seal portion 100 that seals the open part 30A is inserted into the recessed portion (cutout portions 44A and 70A) provided at the inner ring 44 and the pulley 32, with the gap therebetween.

That is, in the X-ray CT apparatus 10 of the embodiment, the seal portion 100 is inserted into the recessed portion (cutout portions 44A and 70A) with the gap therebetween, so that the seal portion 100 and the recessed portion (cutout portions 44A and 70A) constitute the labyrinth seal. As a result, it is possible to suppress the entry of foreign matter such as abrasion powder into the inside of the bearing 30 from the open part 30A and the leakage of a bearing lubricant (grease), with which the inside of the bearing 30 is filled, from the open part 30A.

In addition, the seal portion 110 that seals the open part 30B is inserted into the recessed portion (cutout portions 44B and 66A) provided at the inner ring 44 and the rotation frame 18, with the gap therebetween.

That is, in the X-ray CT apparatus 10 of the embodiment, the seal portion 110 is inserted into the recessed portion (cutout portions 44B and 66A) with the gap therebetween, so that the seal portion 110 and the recessed portion (cutout portions 44B and 66A) constitute the labyrinth seal. As a result, it is possible to suppress the entry of foreign matter such as abrasion powder into the inside of the bearing 30 from the open part 30B and the leakage of the bearing lubricant (grease), with which the inside of the bearing 30 is filled, from the open part 30B.

Although the embodiment according to the present invention has been described above, it is obvious that the present invention is not limited to the above-described embodiment, and various modifications can be made.

### EXPLANATION OF REFERENCES

10: X-ray CT apparatus
16: turn plate
18: rotation frame
20: fixed frame
30: bearing
32: pulley
42: outer ring
44: inner ring
30A: open part
30B: open part
64: opening part
80: slip ring
84: brush device
100: seal portion
110: seal portion

What is claimed is:

1. An X-ray CT apparatus comprising:
a fixed frame;
a rotation frame on which mounted objects including an X-ray source and an X-ray detector are mounted; and
a bearing that supports the rotation frame to be rotatable with respect to the fixed frame,
wherein the bearing includes an outer ring connected to the fixed frame and an inner ring connected to the rotation frame, and
the fixed frame includes a seal portion that seals the bearing.

2. The X-ray CT apparatus according to claim 1,
wherein the seal portion is configured integrally with the fixed frame.

3. The X-ray CT apparatus according to claim 1,
wherein the seal portion is an end part of the fixed frame connected to the outer ring, the end part projecting to face at least one of two open parts on both sides of the bearing in a rotation axis direction.

4. The X-ray CT apparatus according to claim 3, further comprising:
a driving unit including a pulley that rotationally drives the rotation frame,
wherein a recessed portion is provided between the inner ring and the pulley, and
the seal portion is inserted into the recessed portion with a gap therebetween, and the seal portion and the recessed portion constitute a labyrinth seal.

5. The X-ray CT apparatus according to claim 3,
wherein a recessed portion is provided between the inner ring and the rotation frame, and
the seal portion is inserted into the recessed portion with a gap therebetween, and the seal portion and the recessed portion constitute a labyrinth seal.
